(19) United States
(12) Patent Application Publication
(10) Pub. No.: US 2026/0248794 A1
(43) Pub. Date: Aug. 27, 2026

(54) METHODS AND MATERIALS FOR PROMOTING BONE GROWTH

(71) Applicant: Mayo Foundation for Medical Education and Research, Rochester, MN (US)

(72) Inventors: David R. Deyle, Byron, MN (US); Andre J. van Wijnen, Rochester, MN (US); Amel Dudakovic, Rochester, MN (US); Oksana Pichurin, Rochester, MN (US); Margarita E. Carrasco Jeldres, Rochester, MN (US)

(21) Appl. No.: 18/877,409

(22) PCT Filed: Jul. 7, 2023
(86) PCT No.: PCT/US2023/027083

§ 371 Date: Dec. 20, 2024

Related U.S. Application Data
(60) Provisional application No. 63/389,497, filed on Jul. 15, 2022.

(51) Int. Cl. A61K 31/519; A61K 31/444; A61K 31/496; A61P 19/08

(52) U.S. Cl. CPC A61K 31/519; A61K 31/444; A61K 31/496; A61P 19/08

(57) ABSTRACT

This document relates to methods and materials for promoting bone growth. For example, one or more inhibitors of an embryonic ectoderm development (EED) polypeptide can be administered to a mammal (e.g., a human) to promote bone growth within the mammal.

### CROSS-REFERENCE TO RELATED APPLICATIONS

This application claims the benefit of U.S. Patent Application Ser. No. 63/389,497, filed on Jul. 15, 2022. The disclosure of the prior application is considered part of, and is incorporated by reference in, the disclosure of this application.

### TECHNICAL FIELD

This document relates to methods and materials for promoting bone growth. For example, one or more inhibitors of an embryonic ectoderm development (EED) polypeptide can be administered to a mammal (e.g., a human) to promote bone growth within the mammal.

### BACKGROUND INFORMATION

Osteogenesis imperfecta (OI) is a rare, genetic bone disorder that impacts roughly 50,000 patients in the United States, qualifying for orphan disease status with the FDA. Individuals affected with OI have many long-term complications including major skeletal abnormalities, deformities, frequent fractures, pain, and death.

### SUMMARY

This document provides methods and materials for promoting bone growth. For example, one or more inhibitors of an EED polypeptide can be administered to a mammal (e.g., a human) to promote bone growth within the mammal. As demonstrated herein, inhibiting an EED polypeptide can promote bone growth (e.g., can increase bone volume and/or can increase bone density). Having the ability to promote bone growth as described herein (e.g., by administering one or more inhibitors of an EED polypeptide) provides a unique and unrealized opportunity to treat mammals (e.g., humans) having or experiencing bone loss. For example, the ability to promote bone growth can improve the health of patients having a disease, disorder, or condition associated with bone loss.

In general, one aspect of this document features methods for promoting bone growth in a mammal having a bone disorder. The methods can include, or consist essentially of, administering an inhibitor of an EED polypeptide activity to a mammal having a bone disorder, where bone growth increases within the mammal following the administering. The mammal can be an adult human. The mammal can be a juvenile human. The bone disorder can be osteoporosis. The bone disorder can be a genetic bone disorder (e.g., 01). The administering can be an intravenous administration, a subcutaneous administration, or an oral administration. The inhibitor can be A-395 (1-(7-fluoro-2,3-dihydro-1H-inden-1-yl)-N,N-dimethyl-4-[4-(4-methylsulfonylpiperazin-1-yl)phenyl]pyrrolidin-3-amine); MAK683 (N-[(5-fluoro-2,3-dihydro-1-benzofuran-4-yl)methyl]-8-(2-methylpyridin-3-yl)-[1,2,4]triazolo[4,3-c]pyrimidin-5-amine); EED226 (n-(2-furanylmethyl)-8-[4-(methylsulfonyl)phenyl]-1,2,4-triazolo[4,3-c]pyrimidin-5-amine); EEDi-1056 (8-(2,6-dimethylpyridin-3-yl)-N-[(5-fluoro-2,3-dihydro-1-benzofuran-4-yl)methyl]-1-methylsulfonyl-2H-imidazo[1,5-c]pyrimidin-4-ium-5-amine); EEDi-5285 ((3r,4s)-1-[(1s)-7-fluoro-2,3-dihydro-1 h-inden-1-yl]-n,n-dimethyl-4-[4-[4-(methylsulfonyl)-1-piperazinyl]phenyl]-3-pyrrolidinamine); EEDi-5273 (15-[(5-fluoro-2,3-dihydro-1-benzofuran-4-yl)methylamino]-9-propan-2-yl-5-(trifluoromethyl)-4,9,12,14,16-pentazatetracyclo[9.6.1.02,7.014,18]octadeca-1(17),2,4,6,11(18),12,15-heptaen-10-one); or a pharmaceutically acceptable salt thereof. The inhibitor can be FTX-6058 ((S)-12-fluoro-4-(2-methylpyridin-3-yl)-7a,8,13,14-tetrahydro-7H-[1,2,4]triazolo[4′,3′:1,6]pyrido[3,2-b]benzofuro[4,3-fg][1,4]oxazonine), HJM-353, MRTX-2219 ((4S)-8-{4-[(dimethylamino)methyl]-2-methylphenyl}-5-{[(5-fluoro-2,3-dihydro-1-benzofuran-4-yl)methyl]amino}imidazo[1,2-c]pyrimidine-2-carbonitrile)), or a pharmaceutically acceptable salt thereof. The bone growth can increase within about 3 weeks of the administering. The bone growth can increase within about 3 days of the administering. The bone growth can increase by at least 5 percent as measured by bone mineral density (BMD) as compared to a comparable mammal not administered the inhibitor. The method can include identifying the mammal as having the bone disorder. The method can include identifying the mammal as being in need of increased bone growth. The method can include identifying the mammal as being in need of the inhibitor for promoting bone growth. The method can include administering the inhibitor to the mammal from about once per day to about once per month. The method can include administering the inhibitor to the mammal from about once per day to about once per week. The method can include administering from about 5 mg of the inhibitor per kg of body weight of the mammal to about 300 mg of the inhibitor per kg of body weight of the mammal. The method can include administering from about 50 mg of the inhibitor per kg of body weight of the mammal to about 100 mg of the inhibitor per kg of body weight of the mammal. The mammal can not have cancer. The mammal can be a mammal that is not being treated for cancer.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF THE DRAWINGS

FIGS. 1A-1D. H3K27 methylation by the PRC2 complex. FIG. 1A. A schematic showing H3K27 methylation by the PRC2 complex. FIG. 1B. Graphs showing differential expression of Ezh1 and Ezh2 during osteogenic differentiation of mesenchymal stem cells (MSCs). FIG. 1C. A schematic showing how EZH1 can bind to a sub-set of EZH2-target genes. FIG. 1D. A schematic showing that Ezh2 can control initial stages of osteogenic differentiation while Ezh1 can regulate late phases of osteogenic differentiation.

FIG. 2. X-rays of 8-week-old female mice showing higher bone mass with dual loss of Ezh1 and Ezh2 in osteoblasts (X-ray analysis). Mice were Osx-Cre positive, evidencing that the mice were Ezh1 KO/Ezh2 cKO animals having Ezh2 knocked out in bone and Ezh1 knocked out globally.

FIGS. 3A-3B. Higher bone mass with dual loss of Ezh1 and Ezh2 in osteoblasts. FIG. 3A. Microcomputed tomography (microCT or CT) analysis showing higher bone mass with dual loss of Ezh1 and Ezh2 in osteoblasts and pCT re-creations of entire femurs from 8-week-old male mice. FIG. 3B. μCT analysis showing pCT assessment of specific regions of the femurs. Mice were Osx-Cre positive.

FIG. 4. Enhanced biomechanical properties with dual loss of Ezh1 and Ezh2. Assessment of biomechanical properties of 8 week old male mice (n=6 to 10). Mice were Osx-Cre positive. ***=p<0.001.

FIGS. 5A-5F. Inhibition and knock-down of Eed stimulates osteogenesis. Osteoblast differentiation was evaluated using western blotting for H3K27me3 and H3 (FIG. 5A), qPCR for expression of osteogenic genes (FIG. 5B), and alizarin red staining (FIG. 5C) of differentiating MC3T3 cells treated with GSK126 (Ezh2 inhibitor) and A-395 (Eed inhibitor). Osteoblast differentiation was also evaluated using qPCR for expression of PRC2 genes (FIG. 5D), alizarin red staining (FIG. 5E), and qPCR data for expression of osteogenic genes (FIG. 5F) in cells treated with siRNAs targeting Ezh2 and Eed*=p<0.05 relative to control.

FIG. 6. A graph showing that Tmem38b expression increased with Ezh1/Ezh2 loss in osteoblasts. mRNA-Seq of Tmem38b from 3 day old mouse calvarial bone in Ezh1 Ezh2 double knockout (dKO) mice (n=6) compared to the Osx-Cre⁺ WT (CON), Ezh1 only knockout (Ezh1 KO), and Ezh2 only knockout (Ezh2 cKO).

FIG. 7. Graphic representation of EED treatment in OI mice. 5-week old OI mice are treated twice weekly with subcutaneous injection of EED inhibitor or vehicle. Mice are treated for 5 weeks, and are sacrificed and analyzed at 10 weeks of age.

FIG. 8. Photographs of bone density obtained from high resolution μCT. These results demonstrate that a 300 mg/kg does was toxic to OI mice and led to loss of bone density after 5 weeks of treatment. However, when OI mice were treated at a lower dose of 100 mg/kg, bone density increased after 5 weeks of treatment.

FIGS. 9A-9B. Graphs plotting changes in femur length after treatment and mouse weights at 3 weeks of age (3 weeks), at start of EED treatment (5 weeks) and after treatment (10 weeks). OI mice were treated with vehicle and A-395 at 300 mg/kg (FIG. 9A) or 100 mg/kg (FIG. 9B). These results demonstrate that there were no significant changes in femur length or weight after EED treatment.

FIGS. 10A-10B. Graphs plotting changes in bone volume between vehicle OI controls and OI mice treated with 100 mg/kg of A-395 (FIG. 10A) and 300 mg/kg of A-395 (FIG. 10B). These results demonstrate that OI mice treated with 100 mg/kg of A-395 had an increase in overall tissue, bone volume, percent bone per volume, and tissue surface compared to vehicle OI controls. These data indicate an increase in bone volume in 01 mice when treated with A-395. OI mice treated with 300 mg/kg had an increase in tissue volume with no change in tissue surface, but a decrease in bone volume and percent bone per volume indicating a loss of bone with treatment.

FIGS. 11A and 11B. Graphs plotting changes in bone surface area between vehicle 01 controls and OI mice treated with 300 mg/kg of A-395 (FIG. 11A) or 100 mg/kg of A-395 (FIG. 11B). These results demonstrate that treatment with 300 mg/kg of A-395 resulted in a decrease in bone surface, while treatment with 100 mg/kg of A-395 resulted in an increase in bone surface.

FIGS. 12A-12B. Graphs plotting changes in trabecular bone between vehicle OI controls and OI mice treated with 300 mg/kg of A-395 (FIG. 12A) and 100 mg/kg of A-395 (FIG. 12B). These results demonstrate that treatment with 300 mg/kg of A-395 resulted in a decrease in bone surface, while treatment with 100 mg/kg of A-395 resulted in an increase bone. These results demonstrate that treatment with 300 mg/kg of A-395 led to loss of trabecular bone while treatment with 100 mg/kg of A-395 led to an increase in trabecular bone.

FIGS. 13A-13B. Higher bone mass with dual loss of Ezh1 and Ezh2 in OI mice (pCT analysis). μCT assessment of specific regions of the femurs from 8-week-old male mice (FIG. 13A). μCT assessment of bone microstructure showing improvement of bone surface, bone surface density, trabecular number, and trabecular pattern in OI dKO mice compared to OI control (FIG. 13B). All mice were Osx-Cre positive. *P<0.5, **P<0.01, ***P<0.001, ****P<0.0001.

FIGS. 14A-14B. OI mice treated with A-395. μCT assessment of specific regions of the femurs from G610C mice treated with A-395 (FIG. 14A). Assessment of bio-mechanical properties of G610C male mice after treatment with A-395 (FIG. 14B). *=p<0.05.

FIGS. 15A-15B. EED inhibition results in improved bone formation in G610C mice. μCT assessment of specific regions of the femurs from G610C mice treated with MAK683 (FIG. 15A). μCT assessment of bone microstructure showing improvement of multiple bone parameters (FIG. 15B). Treatment with 50 mg/kg and 20 mg/kg showed significant increase in bone density and trabecular structure compared to vehicle treated control. *P<0.5, **P<0.01, ***P<0.001, ****P<0.0001.

FIGS. 16A-16B. EED inhibition prevents bone loss in OVX mice. μCT assessment of specific regions of the femurs from sham or OVX mice treated with vehicle control or MAK683 (FIG. 16A). μCT assessment of bone microstructure showing the prevention of bone loss after ovariectomy. FIG. 16B) Treatment with 20 mg/kg MAK683 showed significant intention of bone density and trabecular number compared to OVX vehicle treated control. *P<0.5, **P<0.01, ***P<0.001, ****P<0.0001.

FIGS. 17A-17M. EED inhibition results in improved bone formation in large cohort of G610C mice. μCT assessment of specific regions of the femurs from G610C mice treated with MAK683 (FIG. 17A). μCT assessment of bone microstructure showing improvement of multiple bone parameters in a cohort of 9 mice in each group (FIGS. 17B-17M). The expanded treatment cohort showed that treatment with 20 mg/kg MAK683 and 10 mg/kg MAK683 showed significant increase in bone density and trabecular structure compared to vehicle treated control. A trend in increased bone quality parameters were seen in OI mice treated with 50 mg/kg MAK683 and 5 mg/kg MAK683. *P<0.5, **P<0.01, ***P<0.001, ****P<0.0001.

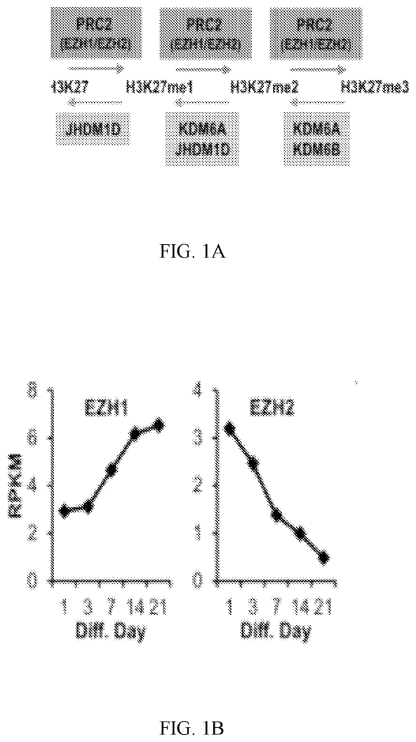

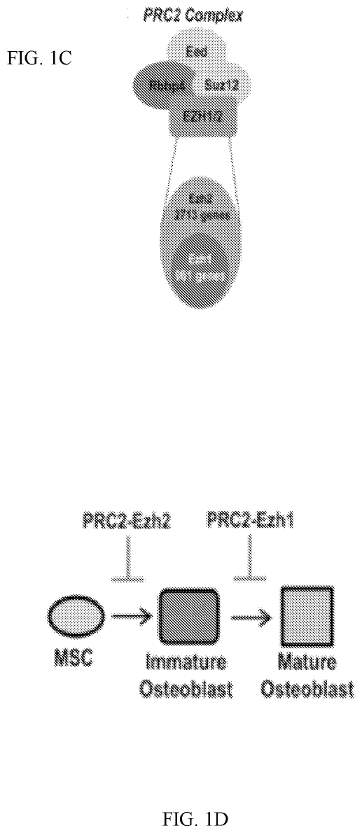

### DETAILED DESCRIPTION

This document provides methods and materials for promoting bone growth. For example, one or more inhibitors of an EED polypeptide can be administered to a mammal (e.g., a human) to promote bone growth within the mammal. In some cases, one or more inhibitors of an EED polypeptide can be administered to a mammal (e.g., a human) having a disease, disorder, or condition associated with bone loss to treat the mammal.

In some cases, one or more inhibitors of an EED polypeptide can be administered to a mammal (e.g., a human) in need thereof (e.g., a human in need of increased bone growth such as a human having a disease, disorder, or condition associated with bone loss) to promote bone growth within the mammal. For example, one or more inhibitors of an EED polypeptide can be administered to a mammal to increase bone growth within the mammal by, for example, 10, 20, 30, 40, 50, 60, 70, 80, 90, 95, or more percent. For example, one or more inhibitors of an EED polypeptide can be administered to a mammal to increase bone growth within the mammal by, for example, at least 1.5 fold (e.g., about 1.5 fold, about 2 fold, about 2.5 fold, about 3 fold, about 3.5 fold, about 4 fold, about 5 fold, about 6 fold, or more).

Any appropriate method can be used to determine the presence of bone growth within the mammal. For example, the presence of one or more bone remodeling markers can be used within a mammal can be used as an indicator of bone formation within that mammal. Examples of bone remodeling markers that can be used as an indicator of bone formation include, without limitation, serum procollagen type 1 N-terminal propeptide (P1NP) polypeptides and urine N-terminal telopeptide of type 1 collagen (NTx) polypeptides.

In some cases, one or more inhibitors of an EED polypeptide can be administered to a mammal (e.g., a human) in need thereof (e.g., a human in need of increased bone growth such as a human having a disease, disorder, or condition associated with bone loss) to increase the bone mineral density (BMD) of one or more bones within the mammal. For example, one or more inhibitors of an EED polypeptide can be administered to a mammal to increase the BMD of one or more bones within the mammal by, for example, 10, 20, 30, 40, 50, 60, 70, 80, 90, 95, or more percent. For example, one or more inhibitors of an EED polypeptide can be administered to a mammal to increase the BMD of one or more bones within the mammal by, for example, at least 1.5 fold (e.g., about 1.5 fold, about 2 fold, about 2.5 fold, about 3 fold, about 3.5 fold, about 4 fold, about 5 fold, about 6 fold, or more).

Any appropriate method can be used to determine the BMD of a bone within a mammal. For example, X-rays, dual-energy X-ray absorptiometry (DEXA), computer tomography scans (e.g., microcomputed tomography (micro-CT) scans), fracture assessments (e.g., fracture assessments using bone surveys), magnetic resonance imaging (MRI), and/or ultrasound scans can be used to determine the BMD of a bone within a mammal.

In some cases, one or more inhibitors of an EED polypeptide can be administered to a mammal (e.g., a human) in need thereof (e.g., a human in need of increased bone growth such as a human having a disease, disorder, or condition associated with bone loss) to increase the bone mineral content (BMC) of one or more bones within the mammal. For example, one or more inhibitors of an EED polypeptide can be administered to a mammal to increase the BMC of one or more bones within the mammal by, for example, 10, 20, 30, 40, 50, 60, 70, 80, 90, 95, or more percent. For example, one or more inhibitors of an EED polypeptide can be administered to a mammal to increase the BMC of one or more bones within the mammal by, for example, at least 1.5 fold (e.g., about 1.5 fold, about 2 fold, about 2.5 fold, about 3 fold, about 3.5 fold, about 4 fold, about 5 fold, about 6 fold, or more).

Any appropriate method can be used to determine the BMC of a bone within a mammal. For example, X-rays, DEXA, CT scans (e.g., micro-CT scans), fracture assessments (e.g., fracture assessments using bone surveys), and/or MRI, ultrasound scans can be used to determine the BMC of a bone within a mammal.

In some cases, one or more inhibitors of an EED polypeptide can be administered to a mammal (e.g., a human) in need thereof (e.g., a human in need of increased bone growth such as a human having a disease, disorder, or condition associated with bone loss) to improve the microarchitecture of one or more bones within the mammal. For example, one or more inhibitors of an EED polypeptide can be administered to a mammal to increase the bone volume fraction of one or more bones within the mammal by, for example, 10, 20, 30, 40, 50, 60, 70, 80, 90, 95, or more percent. In some cases, one or more inhibitors of an EED polypeptide can be administered to a mammal to increase the bone volume fraction of one or more bones within the mammal by, for example, at least 1.5 fold (e.g., about 1.5 fold, about 2 fold, about 2.5 fold, about 3 fold, about 3.5 fold, about 4 fold, about 5 fold, about 6 fold, or more). In another example, one or more inhibitors of an EED polypeptide can be administered to a mammal to increase the trabecular thickness of one or more bones within the mammal by, for example, 10, 20, 30, 40, 50, 60, 70, 80, 90, 95, or more percent. In some cases, one or more inhibitors of an EED polypeptide can be administered to a mammal to increase the trabecular thickness of one or more bones within the mammal by, for example, at least 1.5 fold (e.g., about 1.5 fold, about 2 fold, about 2.5 fold, about 3 fold, about 3.5 fold, about 4 fold, about 5 fold, about 6 fold, or more). In another example, one or more inhibitors of an EED polypeptide can be administered to a mammal to decrease the trabecular separation of one or more bones within the mammal by, for example, 10, 20, 30, 40, 50, 60, 70, 80, 90, 95, or more percent. In some cases, one or more inhibitors of an EED polypeptide can be administered to a mammal to decrease the trabecular separation of one or more bones within the mammal by, for example, at least 1.5 fold (e.g., about 1.5 fold, about 2 fold, about 2.5 fold, about 3 fold, about 3.5 fold, about 4 fold, about 5 fold, about 6 fold, or more).

Any appropriate method can be used to determine the microarchitecture of a bone within a mammal. For example, X-rays, DEXA, CT scans (e.g., micro-CT scans), fracture assessments (e.g., fracture assessments using bone surveys), and/or MRI, ultrasound scans can be used to determine the microarchitecture of a bone within a mammal.

In some cases, one or more inhibitors of an EED polypeptide can be administered to a mammal (e.g., a human) in need thereof (e.g., a human in need of increased bone growth such as a human having a disease, disorder, or condition associated with bone loss) to reduce the rate of bone loss or to prevent bone loss within the mammal. For example, one or more inhibitors of an EED polypeptide can be administered to a mammal to slow bone loss within the mammal by, for example, 10, 20, 30, 40, 50, 60, 70, 80, 90, 95, or more percent. For example, one or more inhibitors of an EED polypeptide can be administered to a mammal to slow bone loss within the mammal by, for example, at least 1.5 fold (e.g., about 1.5 fold, about 2 fold, about 2.5 fold, about 3 fold, about 3.5 fold, about 4 fold, about 5 fold, about 6 fold, or more). In some cases, one or more inhibitors of an EED polypeptide can be administered to a mammal having a disease, disorder, or condition associated with bone loss to prevent further bone loss within the mammal.

Any appropriate mammal can be treated as described herein (e.g., by administering one or more inhibitors of an EED polypeptide to promote bone growth within the mammal). Examples of mammals that can be treated as described herein include, without limitation, humans, non-human primates such as monkeys, horses, bovine species, porcine species, dogs, cats, mice, rats, rabbits, and goats. In some cases, a human (e.g., a human in need of increased bone growth such as a human having a disease, disorder, or condition associated with bone loss) can be treated as described herein. In some cases, an adult mammal (e.g., an adult mammal in need of increased bone growth such as an adult mammal having a disease, disorder, or condition associated with bone loss) can be treated as described herein. For example, an adult human can administered one or more inhibitors of an EED polypeptide to increase bone growth as described herein. For example, a human that is at least 18 years of age can be administered one or more inhibitors of an EED polypeptide to increase bone growth as described herein. In some cases, a juvenile mammal (e.g., a juvenile mammal in need of increased bone growth such as a juvenile mammal having a disease, disorder, or condition associated with bone loss) can be treated as described herein. For example, a juvenile human can administered one or more inhibitors of an EED polypeptide to increase bone growth as described herein. For example, a human that is from about 1 month of age to about 18 years of age (e.g., from about 1 month to about 15 years, from about 1 month to about 12 years, from about 1 month to about 10 years, from about 1 month to about 7 years, from about 1 month to about 5 years, from about 1 month to about 3 years, from about 1 month to about 1 year, from about 1 year to about 18 years, from about 3 years to about 18 years, from about 5 years to about 18 years, from about 8 years to about 18 years, from about 10 years to about 18 years, from about 12 years to about 18 years, from about 15 years to about 18 years, from about 1 year to about 15 years, from about 3 years to about 12 years, from about 5 years to about 10 years, from about 1 year to about 5 years, from about 3 years to about 8 years, from about 5 years to about 10 years, from about 8 years to about 12 years, or from about 10 years to about 15 years of age) can be administered one or more inhibitors of an EED polypeptide to increase bone growth as described herein.

In some cases, a mammal to be treated as described herein (e.g., by administering one or more inhibitors of an EED polypeptide) can have bone loss in one or more injured bones (e.g., can have injury induced bone loss).

In some cases, a mammal to be treated as described herein (e.g., by administering one or more inhibitors of an EED polypeptide to promote bone growth within the mammal) can have one or more diseases, disorders, or conditions associated with bone loss. In some cases, a disease, disorder, or condition associated with bone loss can be a genetic disorder (e.g., a genetic bone disorder). Examples of diseases, disorders, and conditions associated with bone loss that can be treated as described herein include, without limitation, osteoporosis, OI, gnathodiaphyseal dysplasia, dentinogenesis imperfecta, gerodermia osteodysplastica, osteoporosis pseudoglioma syndrome, Hadju-Cheney syndrome, and Cole-Carpenter syndrome.

In some cases, a mammal to be treated as described herein (e.g., by administering one or more inhibitors of an EED polypeptide to promote bone growth within the mammal) can be a mammal (e.g., a human) that does not have cancer. For example, a human that is not being treated for cancer can be treated as described herein (e.g., by administering one or more inhibitors of an EED polypeptide to promote bone growth within the mammal).

One or more inhibitors of an EED polypeptide can be administered to a mammal (e.g., a human) to promote bone growth in any type of bone. In some cases, the methods and materials described herein can promote growth of a cortical bone. In some cases, the methods and materials described herein can promote growth of a cancellous bone. Examples of types of bones that can have bone growth promoted as described herein include, without limitation, long bones, short bones, flat bones, irregular bones, sesamoid bones, and membranous bones.

One or more inhibitors of an EED polypeptide can be administered to a mammal (e.g., a human) to promote bone growth in any bone within a mammal (e.g., a bone in any location within a mammal). In some cases, the methods and materials described herein can be used to promote growth of a bone in the spine of a mammal (e.g., a vertebral bone). In some cases, the methods and materials described herein can be used to promote growth of a bone in an arm of a mammal (e.g., a hand bone). In some cases, the methods and materials described herein can be used to promote growth of a bone in a leg of a mammal (e.g., an ankle bone or a foot bone). In some cases, the methods and materials described herein can be used to promote growth of a bone in the head of a mammal (e.g., a craniofacial bone). In some cases, the methods and materials described herein can be used to promote growth of a bone in the trunk of a mammal (e.g., a pelvic bone, a rib bone, or a sternum bone).

In some cases, the methods described herein can include identifying a mammal (e.g., a human) as being in need of increased bone growth. For example, the methods described herein can include identifying a mammal (e.g., a human) as having a disease, disorder, or condition associated with bone loss. Any appropriate method can be used to identify a mammal as being in need of increased bone growth (e.g., as having bone loss). Examples of methods for identifying a mammal as being in need of increased bone growth (e.g., as having bone loss) include, without limitation, bone density tests, imaging techniques (e.g., X-rays) to determine the proportion of mineral in bones, and laboratory analysis (e.g., for the levels of P1NP polypeptides and/or NTx polypeptides).

A mammal (e.g., a human) can be administered or instructed to self-administer any appropriate inhibitor of an EED polypeptide to promote bone growth as described herein. An inhibitor of an EED polypeptide can inhibit EED polypeptide activity or can inhibit EED polypeptide expression. Examples of inhibitors of EED polypeptide activity include, without limitation, antibodies (e.g., neutralizing antibodies), small molecules that target (e.g., target and bind) to an EED polypeptide, A-395 (1-(7-fluoro-2,3-dihydro-1H-inden-1-yl)-N,N-dimethyl-4-[4-(4-methylsulfonylpiperazin-1-yl)phenyl]pyrrolidin-3-amine); MAK683 (N-[(5-fluoro-2,3-dihydro-1-benzofuran-4-yl)methyl]-8-(2-methylpyridin-3-yl)-[1,2,4]triazolo[4,3-c]pyrimidin-5-amine); EED226 (n-(2-furanylmethyl)-8-[4-(methylsulfonyl)phenyl]-1,2,4-triazolo[4,3-c]pyrimidin-5-amine); EEDi-1056 (8-(2,6-dimethylpyridin-3-yl)-N-[(5-fluoro-2,3-dihydro-1-benzofuran-4-yl)methyl]-1-methylsulfonyl-2H-imidazo[1,5-c]pyrimidin-4-ium-5-amine); EEDi-5285 ((3r,4s)-1-[(1s)-7-fluoro-2,3-dihydro-1h-inden-1-yl]-n,n-dimethyl-4-[4-[4-(methylsulfonyl)-1-piperazinyl]phenyl]-3-pyrrolidinamine); EEDi-5273 (15-[(5-fluoro-2,3-dihydro-1-benzofuran-4-yl)methylamino]-9-propan-2-yl-5-(trifluoromethyl)-4,9,12,14,16-pentazatetracyclo[9.6.1.02,7.014,18]octadeca-1(17),2,4,6,11(18),12,15-heptaen-10-one); FTX-6058 ((S)-12-fluoro-4-(2-methylpyridin-3-yl)-7a,8,13,14-tetrahydro-7H-[1,2,4]triazolo[4′,3′:1,6]pyrido[3,2-b]benzofuro[4,3-fg][1,4]oxazonine); HJM-353; MRTX-2219 ((4S)-8-{4-[(dimethylamino)methyl]-2-methylphenyl}-5-{[(5-fluoro-2,3-dihydro-1-benzofuran-4-yl)methyl]amino}imidazo[1,2-c]pyrimidine-2-carbonitrile)); and pharmaceutically acceptable salts thereof. Examples of inhibitors of an EED polypeptide expression include, without limitation, nucleic acid molecules designed to induce RNA interference of polypeptide expression of an EED polypeptide (e.g., a siRNA molecule or a shRNA molecule), antisense molecules, miRNAs, and CRISPR inhibition. In some cases, an inhibitor of an EED polypeptide that can be used as described herein can be as described elsewhere (see, e.g., Liu et al., RSC Med. Chem., 13:39-53 (2022) at, for example, Table 1; Ma et al., Ann. Oncol., 33(S7):S1122, Abstract 1258P (2022); U.S. Patent Application Publication No. 2020/0360353 at, for example, Table 1 and Example 27; U.S. Pat. No. 9,580,437 at, for example, Table 2 and Table 3; U.S. Patent Publication No. 2022/0227778 at, for example, Table 1; U.S. Pat. No. 10,266,542 at, for example, paragraph [0111] and Table 2; and U.S. Patent Publication No. 2017/0320880 at, for example, paragraphs [0142]-[0307], paragraphs [0583]-[0748], paragraphs [0770]-[0879], Table 5, Table 6, and Table 7).

In some cases, one or more (e.g., one, two, three, four, or more) inhibitors of an EED polypeptide can be formulated into a composition (e.g., a pharmaceutically acceptable composition) for administration to a mammal (e.g., a human). For example, one or more inhibitors of an EED polypeptide can be formulated together with one or more pharmaceutically acceptable carriers (additives), excipients, and/or diluents. Examples of pharmaceutically acceptable carriers, excipients, and diluents that can be used in a composition described herein include, without limitation, cyclodextrins (e.g., beta-cyclodextrins such as KLEPTOSE®), dimethylsulfoxide (DMSO), sucrose, lactose, starch (e.g., starch glycolate), cellulose, cellulose derivatives (e.g., modified celluloses such as microcrystalline cellulose, and cellulose ethers like hydroxypropyl cellulose (HPC) and cellulose ether hydroxypropyl methylcellulose (HPMC)), xylitol, sorbitol, mannitol, gelatin, polymers (e.g., polyvinylpyrrolidone (PVP), polyethylene glycol (PEG), crosslinked polyvinylpyrrolidone (crospovidone), carboxymethyl cellulose, polyethylene-polyoxypropylene-block polymers, and crosslinked sodium carboxymethyl cellulose (croscarmellose sodium)), titanium oxide, azo dyes, silica gel, fumed silica, tale, magnesium carbonate, vegetable stearin, magnesium stearate, aluminum stearate, stearic acid, antioxidants (e.g., vitamin A, vitamin E, vitamin C, retinyl palmitate, and selenium), citric acid, sodium citrate, parabens (e.g., methyl paraben and propyl paraben), petrolatum, dimethyl sulfoxide, mineral oil, serum proteins (e.g., human serum albumin), glycine, sorbic acid, potassium sorbate, water, salts or electrolytes (e.g., saline such as phosphate buffered saline, protamine sulfate, disodium hydrogen phosphate, potassium hydrogen phosphate, sodium chloride, and zinc salts), colloidal silica, magnesium trisilicate, polyacrylates, waxes, wool fat, lecithin, and corn oil.

In some cases, when a composition containing one or more (e.g., one, two, three, four, or more) inhibitors of an EED polypeptide is administered to a mammal (e.g., a human), the composition can be designed for oral or parenteral (including, without limitation, subcutaneous and intravenous injections) administration to the mammal. Compositions suitable for oral administration include, without limitation, liquids, tablets, capsules, pills, powders, gels, and granules. Compositions suitable for parenteral administration include, without limitation, aqueous and non-aqueous sterile injection solutions that can contain anti-oxidants, buffers, bacteriostats, and solutes that render the formulation isotonic with the blood of the intended recipient.

In some cases, a composition containing one or more (e.g., one, two, three, four, or 30 more) inhibitors of an EED polypeptide can be in the form of a sterile injectable suspension (e.g., a sterile injectable aqueous or oleaginous suspension). This suspension may be formulated using, for example, suitable dispersing or wetting agents (such as, for example, Tween 80) and suspending agents. The sterile injectable preparation can be a sterile injectable solution or suspension in a non-toxic parenterally-acceptable diluent or solvent, for example, as a solution in 1,3-butanediol. Examples of acceptable vehicles and solvents that can be used include, without limitation, saline, mannitol, water, Ringer's solution, and isotonic sodium chloride solution. In addition, sterile, fixed oils can be used as a solvent or suspending medium. In some cases, a bland fixed oil can be used such as synthetic mono- or di-glycerides.

In some cases, a composition containing one or more (e.g., one, two, three, four, or more) inhibitors of an EED polypeptide can be presented in unit-dose or multi-dose containers, for example, sealed ampules and vials, and may be stored in a freeze dried (lyophilized) condition requiring only the addition of the sterile liquid carrier, for example water for injections, immediately prior to use.

A composition containing one or more (e.g., one, two, three, four, or more) inhibitors of an EED polypeptide can be administered to a mammal (e.g., a human) in any appropriate amount (e.g., any appropriate dose). An effective amount of a composition containing one or more inhibitors of an EED polypeptide can be any amount that can promote bone growth as described herein without producing significant toxicity to the mammal. In cases where an inhibitor of an EED polypeptide is A-395, an effective amount of the inhibitor can be from about 50 milligrams per kilogram body weight (mg/kg) to about 100 mg/kg (e.g., from about 50 mg/kg to about 90 mg/kg, from about 50 mg/kg to about 80 mg/kg, from about 50 mg/kg to about 70 mg/kg, from about 50 mg/kg to about 60 mg/kg, from about 60 mg/kg to about 100 mg/kg, from about 70 mg/kg to about 100 mg/kg, from about 80 mg/kg to about 100 mg/kg, from about 90 mg/kg to about 100 mg/kg, from about 60 mg/kg to about 90 mg/kg, from about 70 mg/kg to about 80 mg/kg, from about 60 mg/kg to about 70 mg/kg, or from about 80 mg/kg to about 90 mg/kg). In cases where an inhibitor of an EED polypeptide is MAK683, an effective amount of the inhibitor can be from about 5 mg/kg to about 300 mg/kg (e.g., from about 5 mg/kg to about 250 mg/kg, from about 5 mg/kg to about 200 mg/kg, from about 5 mg/kg to about 150 mg/kg, from about 5 mg/kg to about 100 mg/kg, from about 5 mg/kg to about 50 mg/kg, from about 5 mg/kg to about 25 mg/kg, from about 25 mg/kg to about 300 mg/kg, from about 50 mg/kg to about 300 mg/kg, from about 100 mg/kg to about 300 mg/kg, from about 150 mg/kg to about 300 mg/kg, from about 200 mg/kg to about 300 mg/kg, from about 250 mg/kg to about 300 mg/kg, from about 25 mg/kg to about 250 mg/kg, from about 50 mg/kg to about 200 mg/kg, from about 100 mg/kg to about 150 mg/kg, from about 25 mg/kg to about 50 mg/kg, from about 50 mg/kg to about 100 mg/kg, from about 150 mg/kg to about 200 mg/kg, or from about 200 mg/kg to about 250 mg/kg). In cases where an inhibitor of an EED polypeptide is EED226, an effective amount of the inhibitor can be from about 5 mg/kg to about 300 mg/kg (e.g., from about 5 mg/kg to about 250 mg/kg, from about 5 mg/kg to about 200 mg/kg, from about 5 mg/kg to about 150 mg/kg, from about 5 mg/kg to about 100 mg/kg, from about 5 mg/kg to about 50 mg/kg, from about 5 mg/kg to about 25 mg/kg, from about 25 mg/kg to about 300 mg/kg, from about 50 mg/kg to about 300 mg/kg, from about 100 mg/kg to about 300 mg/kg, from about 150 mg/kg to about 300 mg/kg, from about 200 mg/kg to about 300 mg/kg, from about 250 mg/kg to about 300 mg/kg, from about 25 mg/kg to about 250 mg/kg, from about 50 mg/kg to about 200 mg/kg, from about 100 mg/kg to about 150 mg/kg, from about 25 mg/kg to about 50 mg/kg, from about 50 mg/kg to about 100 mg/kg, from about 150 mg/kg to about 200 mg/kg, or from about 200 mg/kg to about 250 mg/kg). In cases where an inhibitor of an EED polypeptide is EEDi-1056, an effective amount of the inhibitor can be from about 5 mg/kg to about 300 mg/kg (e.g., from about 5 mg/kg to about 250 mg/kg, from about 5 mg/kg to about 200 mg/kg, from about 5 mg/kg to about 150 mg/kg, from about 5 mg/kg to about 100 mg/kg, from about 5 mg/kg to about 50 mg/kg, from about 5 mg/kg to about 25 mg/kg, from about 25 mg/kg to about 300 mg/kg, from about 50 mg/kg to about 300 mg/kg, from about 100 mg/kg to about 300 mg/kg, from about 150 mg/kg to about 300 mg/kg, from about 200 mg/kg to about 300 mg/kg, from about 250 mg/kg to about 300 mg/kg, from about 25 mg/kg to about 250 mg/kg, from about 50 mg/kg to about 200 mg/kg, from about 100 mg/kg to about 150 mg/kg, from about 25 mg/kg to about 50 mg/kg, from about 50 mg/kg to about 100 mg/kg, from about 150 mg/kg to about 200 mg/kg, or from about 200 mg/kg to about 250 mg/kg). In cases where an inhibitor of an EED polypeptide is EEDi-5285, an effective amount of the inhibitor can be from about 5 mg/kg to about 300 mg/kg (e.g., from about 5 mg/kg to about 250 mg/kg, from about 5 mg/kg to about 200 mg/kg, from about 5 mg/kg to about 150 mg/kg, from about 5 mg/kg to about 100 mg/kg, from about 5 mg/kg to about 50 mg/kg, from about 5 mg/kg to about 25 mg/kg, from about 25 mg/kg to about 300 mg/kg, from about 50 mg/kg to about 300 mg/kg, from about 100 mg/kg to about 300 mg/kg, from about 150 mg/kg to about 300 mg/kg, from about 200 mg/kg to about 300 mg/kg, from about 250 mg/kg to about 300 mg/kg, from about 25 mg/kg to about 250 mg/kg, from about 50 mg/kg to about 200 mg/kg, from about 100 mg/kg to about 150 mg/kg, from about 25 mg/kg to about 50 mg/kg, from about 50 mg/kg to about 100 mg/kg, from about 150 mg/kg to about 200 mg/kg, or from about 200 mg/kg to about 250 mg/kg). In cases where an inhibitor of an EED polypeptide is EEDi-5273, an effective amount of the inhibitor can be from about 5 mg/kg to about 300 mg/kg (e.g., from about 5 mg/kg to about 250 mg/kg, from about 5 mg/kg to about 200 mg/kg, from about 5 mg/kg to about 150 mg/kg, from about 5 mg/kg to about 100 mg/kg, from about 5 mg/kg to about 50 mg/kg, from about 5 mg/kg to about 25 mg/kg, from about 25 mg/kg to about 300 mg/kg, from about 50 mg/kg to about 300 mg/kg, from about 100 mg/kg to about 300 mg/kg, from about 150 mg/kg to about 300 mg/kg, from about 200 mg/kg to about 300 mg/kg, from about 250 mg/kg to about 300 mg/kg, from about 25 mg/kg to about 250 mg/kg, from about 50 mg/kg to about 200 mg/kg, from about 100 mg/kg to about 150 mg/kg, from about 25 mg/kg to about 50 mg/kg, from about 50 mg/kg to about 100 mg/kg, from about 150 mg/kg to about 200 mg/kg, or from about 200 mg/kg to about 250 mg/kg). The effective amount can remain constant or can be adjusted as a sliding scale or variable dose depending on the mammal's response to treatment. Various factors can influence the actual effective amount used for a particular application. For example, the frequency of administration, duration of treatment, use of multiple treatment agents, route of administration, and/or severity of bone loss in the mammal being treated may require an increase or decrease in the actual effective amount administered.

A composition containing one or more (e.g., one, two, three, four, or more) inhibitors of an EED polypeptide can be administered to a mammal (e.g., a human) at any appropriate frequency. The frequency of administration can be any frequency that can promote bone growth in a mammal without producing significant toxicity to the mammal. For example, the frequency of administration can be from about twice a day to about one every other day, from about once a day to about once a week, from about once a day to about once a month, from about once a week to about once a month, from about twice a month to about once a month, from about once a month to about once every three months, from about once every 3 months to about once every 6 months, or from about once every 6 months to about once a year. The frequency of administration can remain constant or can be variable during the duration of treatment. As with the effective amount, various factors can influence the actual frequency of administration used for a particular application. For example, the effective amount, duration of treatment, use of multiple treatment agents, and/or route of administration may require an increase or decrease in administration frequency.

A composition containing one or more (e.g., one, two, three, four, or more) inhibitors of an EED polypeptide can be administered to a mammal (e.g., a human) for any appropriate duration. An effective duration for administering or using a composition containing one or more inhibitors of an EED polypeptide can be any duration that can promote bone growth in a mammal without producing significant toxicity to the mammal. For example, the effective duration can vary from several weeks to several months, from several months to several years, or from several years to a lifetime. Multiple factors can influence the actual effective duration used for a particular treatment. For example, an effective duration can vary with the frequency of administration, effective amount, use of multiple treatment agents, and/or route of administration.

In some cases, methods for promoting bone growth within a mammal (e.g., a human) as described herein (e.g., by administering one or more inhibitors of an EED polypeptide) can include administering to the mammal one or more (e.g., one, two, three, four, or more) inhibitors of an EED polypeptide as the sole active ingredient to promote bone growth in the mammal. For example, a composition containing one or more inhibitors of an EED polypeptide can include the one or more inhibitors of an EED polypeptide as the sole active ingredient(s) in the composition that is effective to promote bone growth within a mammal.

In some cases, methods for promoting bone growth within a mammal (e.g., a human) as described herein (e.g., by administering one or more inhibitors of an EED polypeptide) also can include administering to the mammal one or more (e.g., one, two, three, four, five or more) additional agents used to promote bone growth to the mammal and/or performing one or more therapies used to promote growth on the mammal. For example, a combination therapy used to promote bone growth can include administering to the mammal (e.g., a human) one or more inhibitors of an EED polypeptide described herein and administering one or more (e.g., one, two, three, four, five or more) additional agents used to promote bone growth. In some cases, an agent that can be used to promote bone growth can be a hormone. In some cases, an agent that can be used to promote bone growth can be a TGF-beta inhibitor. In some cases, an agent that can be used to promote bone growth can be a bisphosphonate. Examples of additional agents that can be administered to a mammal to promote bone growth within the mammal include, without limitation, teriparatide, abaloparatide, romosozumab, fresolimumab, SAR439459, setrusumab, denosumab (e.g., PROLIA® and XGEVA®), growth hormone, and any combinations thereof. In cases where one or more inhibitors of an EED polypeptide are used in combination with additional agents used to promote bone growth, the one or more additional agents can be administered at the same time (e.g., in a single composition containing both one or more inhibitors of an EED polypeptide and the one or more additional agents) or independently. For example, one or more inhibitors of an EED polypeptide described herein can be administered first, and the one or more additional agents administered second, or vice versa.

In some cases, a combination therapy used to promote bone growth can include administering to the mammal (e.g., a human) one or more (e.g., one, two, three, four, or more) inhibitors of an EED polypeptide described herein and performing one or more (e.g., one, two, three, four, five or more) additional therapies used to promote bone growth on the mammal. Examples of therapies used to promote bone growth include, without limitation, physical therapies (e.g., exercise), casting, and/or bone fixation. In cases where one or more inhibitors of an EED polypeptide described herein are used in combination with one or more additional therapies used to promote bone growth, the one or more additional therapies can be performed at the same time or independently of the administration of one or more inhibitors of an EED polypeptide described herein. For example, one or more inhibitors of an EED polypeptide described herein can be administered before, during, or after the one or more additional therapies are performed.

In cases where one or more inhibitors of an EED polypeptide are administered to a mammal (e.g., a human in need of increased bone growth such as a human having a disease, disorder, or condition associated with bone loss) to treat the mammal, the methods for promoting bone growth within a mammal (e.g., a human) as described herein (e.g., by administering one or more inhibitors of an EED polypeptide to promote bone growth within the mammal) also can include administering to the mammal one or more (e.g., one, two, three, four, five or more) additional agents used to treat bone loss to the mammal. For example, a combination therapy used to treat a disease, disorder, or condition associated with bone loss can include administering to the mammal (e.g., a human) one or more inhibitors of an EED polypeptide described herein and one or more (e.g., one, two, three, four, five or more) agents used to treat bone loss. In some cases, an agent that can be used to treat bone loss can be a bisphosphonate. In some cases, an agent that can be used to treat bone loss can be a hormone. Examples of agents that can be used to treat bone loss include, without limitation, alendronate (e.g., BINOSTO® and FOSAMAX®), risedronate (e.g., ACTONEL® and ATELVIA®), ibandronate (e.g., BONIVA®), zoledronic acid (e.g., RECLAST® and ZOMETA®), denosumab (e.g., PROLIA® and XGEVA®), estrogen, raloxifene (e.g., EVISTA®), teriparatide (e.g., FORTEO®), abaloparatide (e.g., TYMLOS®), and romosozumab (EVENITY®). In cases where one or more inhibitors of an EED polypeptide are used in combination with additional agents used to treat bone loss, the one or more additional agents can be administered at the same time (e.g., in a single composition containing both one or more inhibitors of an EED polypeptide and the one or more additional agents) or independently. For example, one or more inhibitors of an EED polypeptide described herein can be administered first, and the one or more additional agents administered second, or vice versa.

The invention will be further described in the following examples, which do not limit the scope of the invention described in the claims.

### EXAMPLES

### Example 1. Skeletal Phenotype of Oi Mice after Dual Loss of EZH1/EZH2

### Materials and Methods

### Ezh1/Ezh2 Knockout Mice

Mice harboring two copies of the Ezh2 allele with loxP sites flanking the SET domain (Ezh2^{fl/fl}) were obtained from the Mutant Mouse Regional Resource Centre (B6;129P2-Ezh2tm1Tara/Mmnc, University of North Carolina, Chapel Hill, NC). Osx-Cre⁺ mice were obtained from Jackson Laboratory (Bar Harbor, ME). Mice were mated to generate the mouse line: Ezh1^{KO/KO}: Ezh2^{flox/flox}: Osx-Cre⁺ (Ezh1 KO/Ezh2 cKO) that conditionally knocks out Ezh2 in bone and globally knocks out Ezh1.

### Radiographic and Micro-CT Analysis

Radiographic analysis was performed using a Faxitron X-ray imaging cabinet (Faxitron Bioptics, Tucson, AZ). Quantitative analysis of the femur was performed using a dual-energy x-ray absorptiometry (Lunar PIXImus2), micro-computed tomography (microCT or μCT) (Scanco Medical AG, μCT35). A 10.5 μm voxel size using a threshold of 220 was applied to all scans at high resolution. Two-dimensional data from scanned slices were used for 3D analysis of morphometric parameters of trabecular bone mass and microarchitecture.

### Mechanical Testing

Three-point bend fixture was mounted on a servohydrauilic mechanical testing frame (#312, MTS Systems) instrumented with a 100-N capacity load cell (#3397-25, Leebow Products). Femurs were be mounted on supports spanning 8 mm. Point loading was applied to the femur mid-shaft allowing the bones to flex about an axis aligned with the medial-lateral line. Loading was applied under displacement control at a rate of 20 mm/minute until fracture. Force and displacement data were be sampled at 256 Hz. Peak load and peak displacement was be quantified. The stiffness was be calculated from the slope of the linear region of the force displacement curve.

### Results

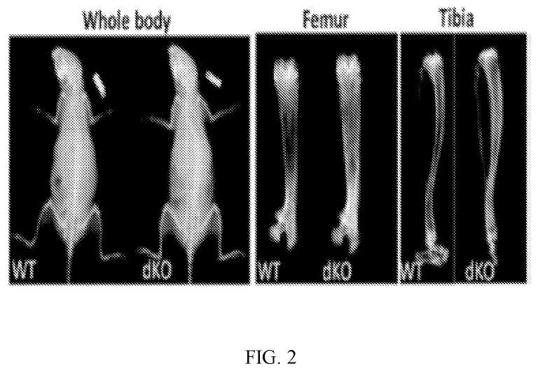

Dual loss of Ezh1 and Ezh2 in osteoblasts produced mice with higher bone mass and strength. Mice in which both Ezh1 (global knock-out (KO)) and Ezh2 (Osx-Cre cKO) were ablated in osteoblasts were used. Double knockout mice (dKO) were born at expected ratios and were physically indistinguishable from wild-type (WT) cohorts (FIG. 2, left panel). Measured by x-ray analysis, the first cohort of dKO mice exhibited higher bone mass in femurs (FIG. 2, middle panel) and tibias (FIG. 2, right panel) when compared to WT littermates.

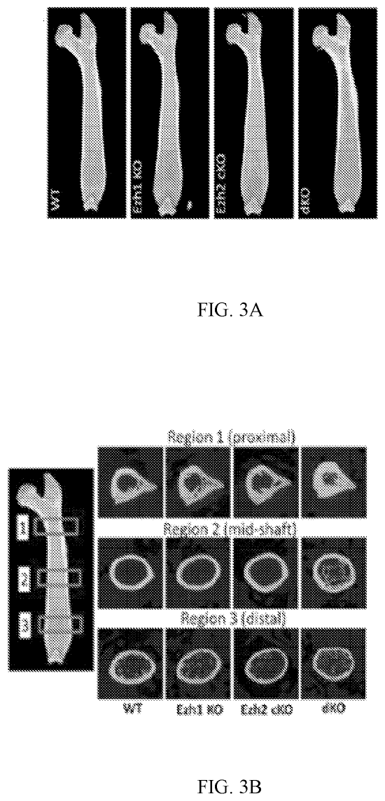

μCT analysis demonstrated robust bone formation within the femurs of dKO mice when compared to WT, Ezh1 KO, and Ezh2 cKO mice (FIGS. 3A and 3B). The results indicated a significant thickening of cortical bone and enhancement of trabecular bone. Extensive bone formation within bone cavities was observed during marrow removal of femurs and tibias in both male and female dKO mice.

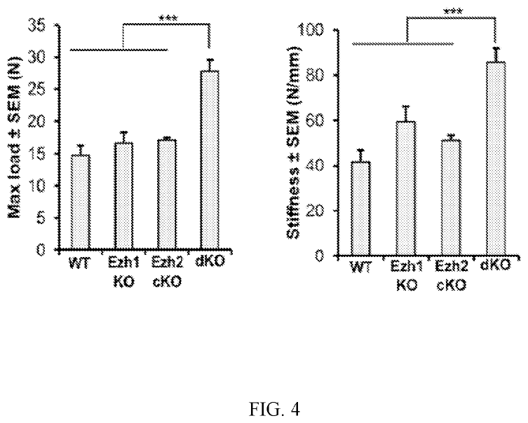

Results indicated that dual loss of Ezh1 and Ezh2 (but not of either Ezh1 or Ezh2 alone) significantly enhanced biomechanical properties (max load and stiffness) as determined by biomechanical testing (three-point bend) in femurs derived from eight-week-old male mice (FIG. 4). The results indicated single KO of either Ezh1 or Ezh2 resulted in either no or negative bone phenotype, while combined loss showed a remarkably robust bone phenotype which increased mechanical strength.

### Example 2. Inhibition of PRC2 Complex to Stimulate Bone Formation

### Materials and Methods

### G610C Mouse Model

Five-week-old OI mice (mice harboring the Col1a2 c.2098G>T p.Gly700Cys allele [Is this accurate?]) were subcutaneously injected with DMSO (vehicle) or EED inhibitor A-395 (Sigma) at concentrations of either 100 mg/kg or 300 mg/kg twice weekly for 5 weeks.

### MC3T3 Cell Culture

MC3T3 sc4 murine calvarial osteoblasts were purchased from American Type Culture Collection and maintained in α-minimal essential medium without ascorbic acid (Gibco) containing 10% fetal bovine serum (Atlanta Biologicals), 100 units/mL penicillin, and 100 g/mL streptomycin (Gibco).

### Osteogenic Differentiation

For osteogenic differentiation, MC3T3s were seeded in respective maintenance medium at a density of 10,000 cells/cm². Next day, osteogenic medium supplemented with vehicle, GSK126 (Xcess Biociences Inc.) or A-395 (Sigma) was added to the cells. Osteogenic medium for MC3T3 cells consisted of 50 μg/mL ascorbic acid (Sigma) and 4 mm β-glycerol phosphate (Sigma). Three days later, old medium was replaced with a fresh batch of osteogenic medium supplemented with vehicle, GSK126, or A-395. On day 6 of differentiation, fresh osteogenic medium without supplements was added and replenished every 2 to 3 days. On day 14, cells were fixed in 10% neutral buffered formalin and stained with 2% alizarin red to visualize calcium deposition.

### Real-Time Reverse Transcriptase PCR (RT-qPCR)

Total RNA was isolated using the Direct-zol™ RNA kit (Zymo Research) and quantified using the NanoDrop 2000 spectrophotometer (Thermo Fischer Scientific). RNA was then reverse transcribed into cDNA by the SuperScript III First-Strand Synthesis System (Invitrogen). Transcript levels were then measured using the 2^{ΔΔct} method and normalized GAPDH (set at 100), a housekeeping gene.

### Western Blotting

MC3T3 cells (4,000 cells/cm²) were plated in 6-well plates in maintenance medium. Cells were treated with vehicle or EED inhibitor (A-395) at 0.02, 0.05, 0.1, 1, 5, or 10 mM. Cells were lysed in radioimmunoprecipitation buffer (150 mm NaCl, 50 mm Tris, pH 7.4, 1% sodium deoxycholate, 0.1% sodium dodecyl sulfate, 1% Triton X-100) supplemented with protease inhibitor mixture (Sigma) and phenylmethylsulfonyl fluoride (Sigma). Lysates were cleared by centrifugation. Protein concentrations were determined by the DC Protein Assay (Bio-Rad). Proteins were resolved by SDS-PAGE and transferred to polyvinylidene difluoride membranes. After blocking in 5% nonfat dry milk for 45 minutes at room temperature, primary antibodies were added overnight at 4° C., followed by secondary antibodies for 1 hour at room temperature. Proteins were visualized using an ECL Prime detection kit. Primary antibodies used were: Tubulin (1:10,000; Mayo Clinic), H3 (1:10,000; 05-928; Millipore), H3K27me3 (1:5,000; 17-622; Millipore), and EZH2 (1:10,000; 5246; Cell Signaling).

### MicroCT Analysis

Femurs of injected mice were scanned by μCT Skyscan 1076 (Brunker) at the maximal potencial 60 kV and 167 μA with 0.25 mm thick aluminum filter and at the voxel resolution of 9 μm. The μCT scans were performed over 360° of total rotation with each angular rotation step of 0.7°. The reconstruction, performed using the NRecon software package (Skycan), were based on the Feldkamp algorithm and resulted in axial grayscale image. The trabecular region of interest were selected with reference to the growth plate. The 2D images were created using CTAn siftware package (Skyscan). The 3D μCT models of each femur were created using a 3D reconstruction software package (Skyscan).

### Results

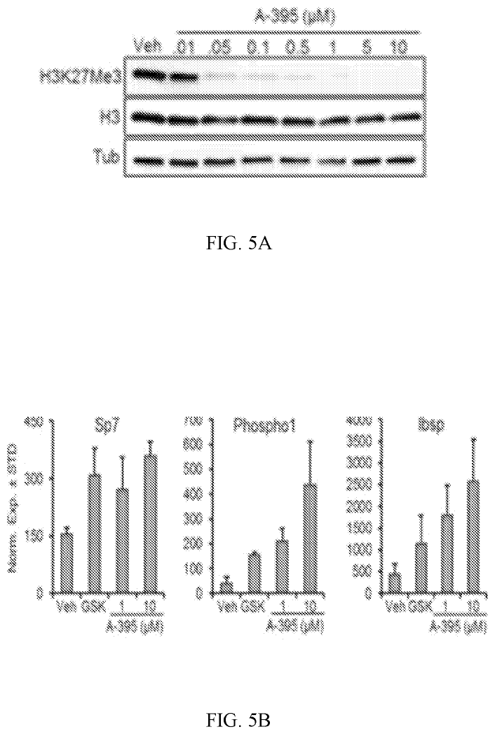

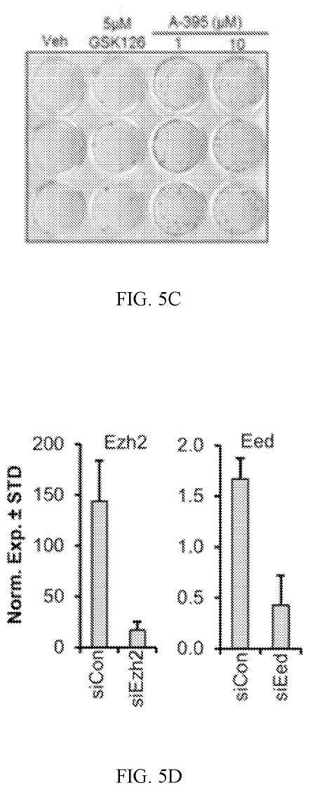

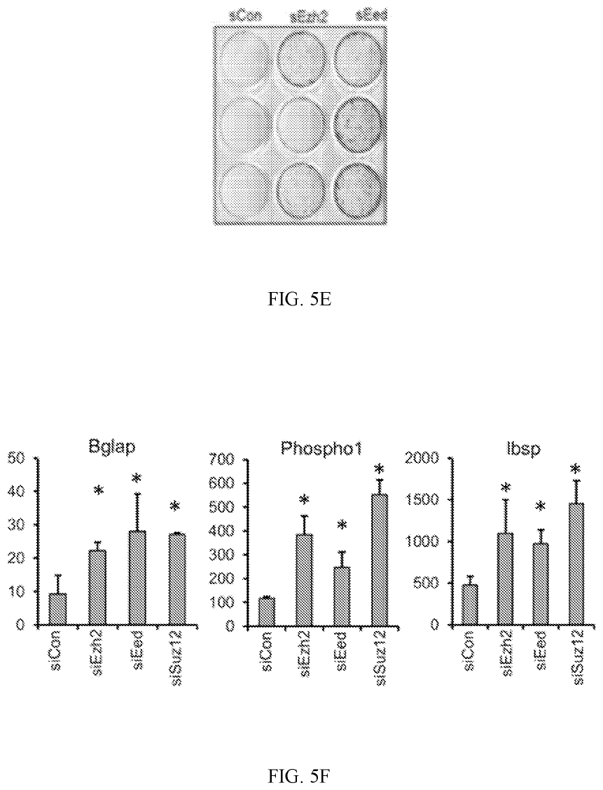

PRC2 complex disruption was assessed by an inhibition or a knockdown of EED in differentiated MC3T3 pre-osteoblasts. The EED inhibitors A-395 and EED226 each reduced H3K27me3 in a concentration-dependent manner in MC3T3 cells (FIG. 5A). MC3T3 osteogenesis was monitored by RT-qPCR of osteogenic genes (FIG. 5B) and alizarin red staining (FIG. 5C). siRNA knockdown of Eed also stimulated differentiating MC3T3 cells osteogenesis as monitored by expression of osteogenic genes (FIG. 5D) and alizarin red staining (FIG. 5E). Inhibition and knockdown of Eed was more potent at stimulating osteogenic differentiation when compared to inhibition and knockdown of Ezh2 (e.g., compare FIG. 5C and FIG. 5E). The results indicated that targeting both PRC2-Ezh1 and PRC2-Ezh2 generated higher bone mass.

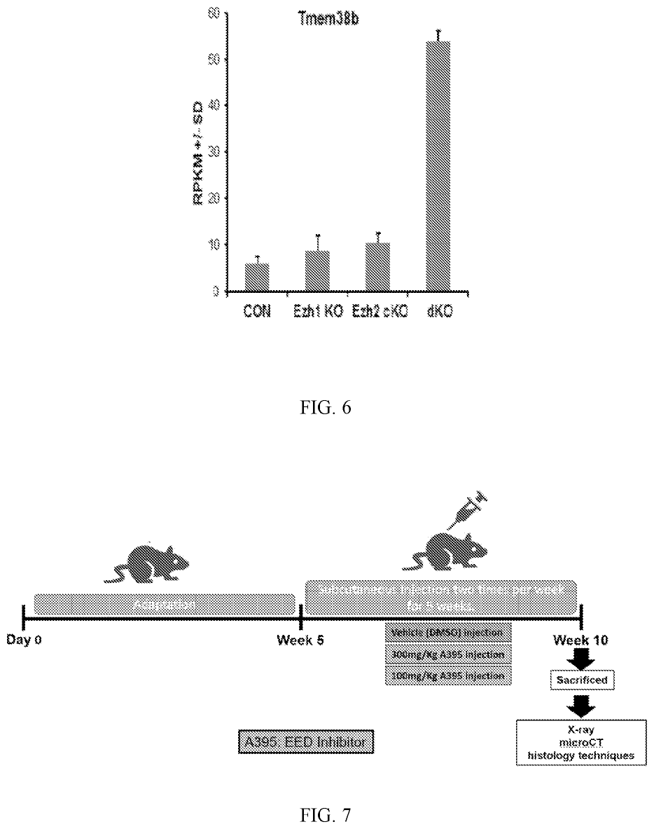

To determine whether Tmem38b expression increased with Ezh1/Ezh2 loss in osteoblasts, expression of Tmem38b in calvarial bone from 3-day old Ezh1 Ezh2 dKO mice was examined. EZH1 and EZH2 inhibition upregulated Tmem38b gene expression (FIG. 6). These results indicated that iMEM38B upregulation can be used to stimulate bone formation.

The method for treating OI mice with and EED inhibitor is shown in FIG. 7. 5-week-old OI mice were treated twice weekly with a subcutaneous injection of either vehicle (DMSO) or EED inhibitor A-395 had concentrations of 300 mg/kg or 100 mg/kg. These mice were treated for five weeks and then analyzed for changes in bone structure.

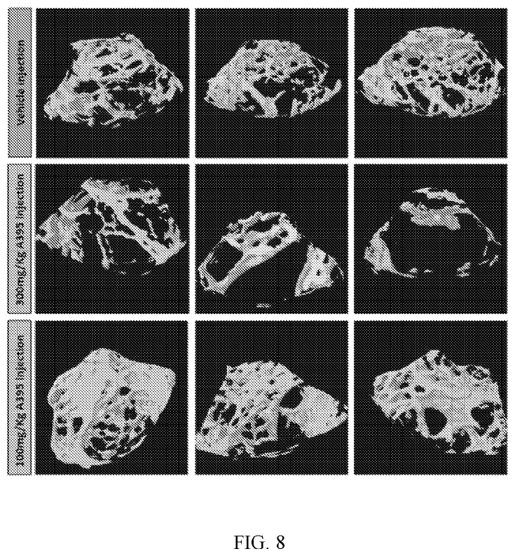

μCT analysis demonstrated OI mice treated with the high dose (300 mg/kg) of A-395 had loss of bone mineralization. OI mice treated with the lower dose (100 mg/kg) of A-395 showed robust bone formation within the femurs (FIG. 8). These results indicate that the lower dose of A-395 resulted in an increase in bone formation or as the higher dose was toxic to the mouse and resulted in loss of bone mineralization.

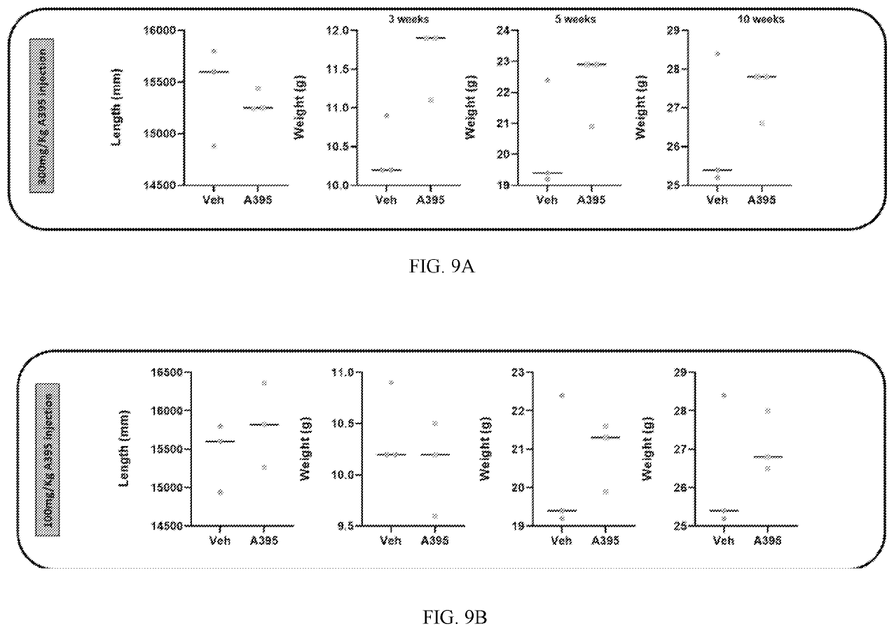

Quantitative analysis of the overall mouse phenotype showed that there were no significant differences in the femur length between treated and untreated animals. There was a trend for increased weights in animals treated with the EED inhibitor (FIGS. 9A and 9B). Since the changes in weight were seen prior to treatment, this can be attributed to variation within the mass population.

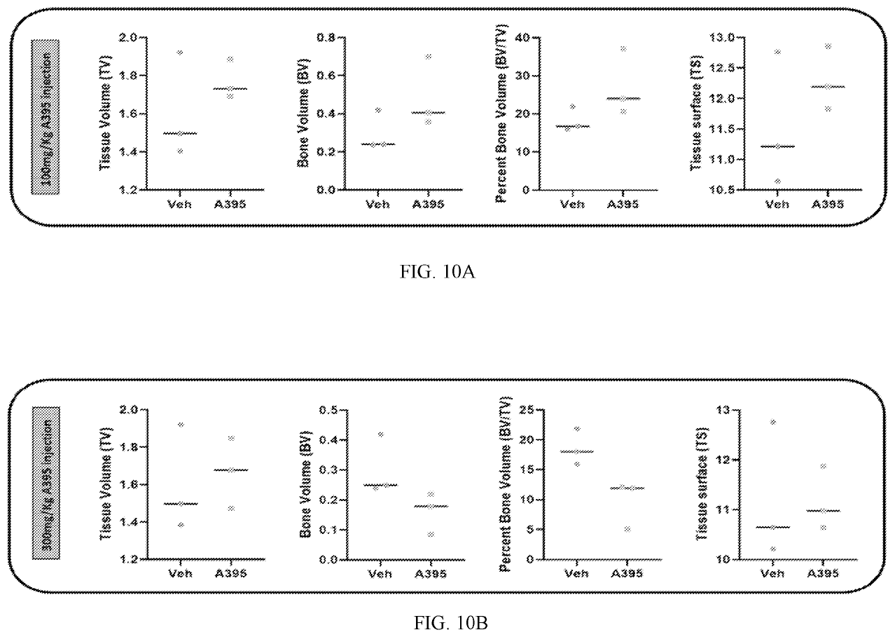

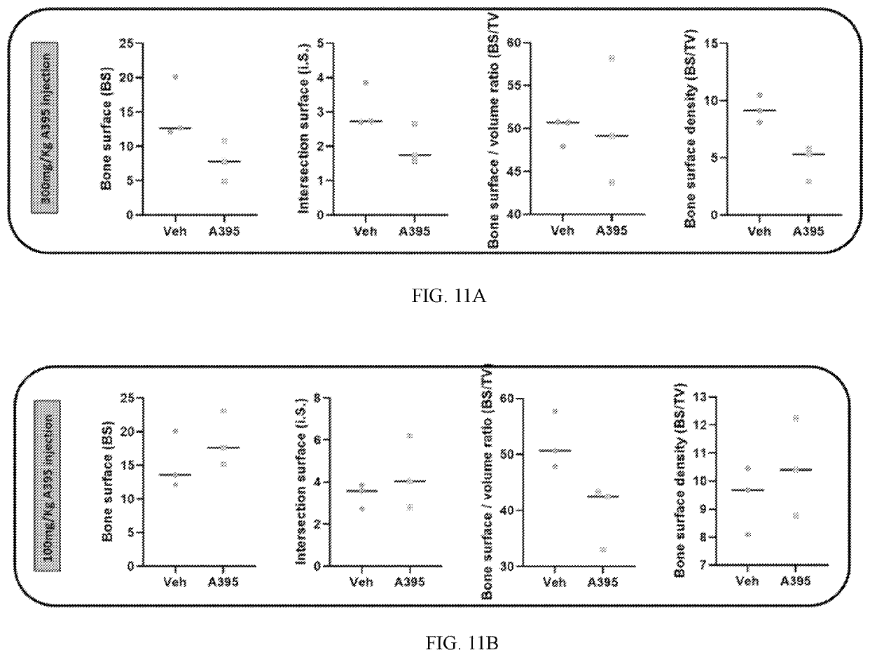

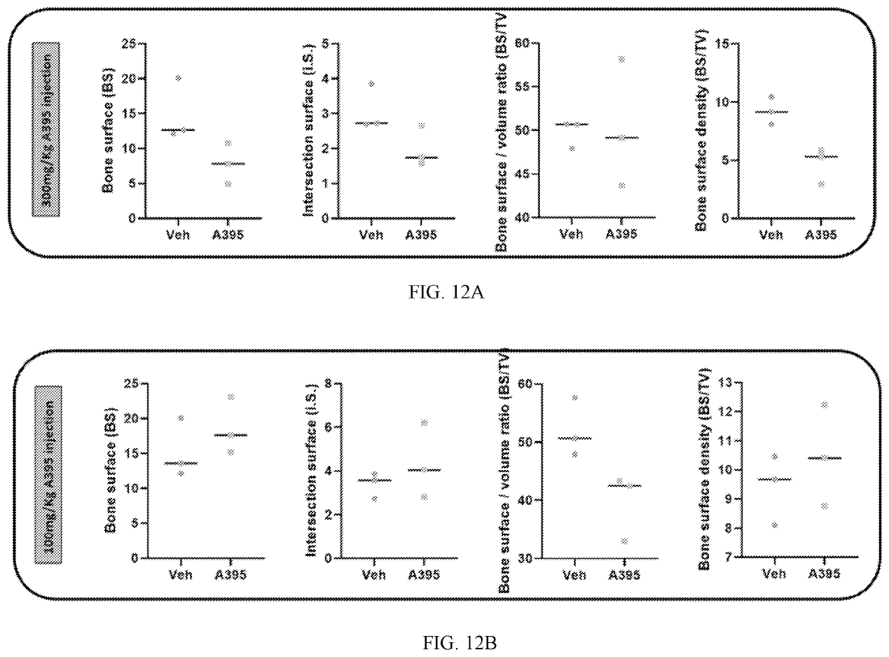

μCT results showed that total bone tissue volume for mice treated with A-395 or increased however while bone volumes, percentage of bone volume, and tissue surface were increased with treatment at 100 mg/kg they were decreased with treatment at 300 mg/kg (FIGS. 10A and 10B). In addition, bone surface, intersection surface, and bone density were down and bone surface to volume ratio was unchanged in the OI mice treated at 300 mg/kg (FIG. 11A), while 01 mice treated at 100 mg/kg had increase in bone surface, and bone surface density with no change in intersection surface and a decrease in the bone surface to volume ratio (FIG. 11B). Further trabecular analysis showed that OI mice treated at 300 mg/kg had a slight decrease in their trabecular thickness and trabecular pattern with increase intravascular separation and decrease in trabecular number (FIG. 12A). OI mice treated at 100 mg/kg saw an increase in trabecular thickness and number with a slight increase intravascular separation and a decrease and trabecular pattern (FIG. 12B).

TABLE 1
Weight of OI mice treated with A-395.

| Mouse | Weigh at 3 | Weight at 5 | Weight at 10 | |
|---|---|---|---|---|
| ID | weeks | weeks | weeks | Treatment |
| D1586 | 10.2 | 19.2 | 25.4 | Veh |
| D1590 | 10.9 | 22.4 | 28.4 | Veh |
| D1587 | 10.2 | 19.4 | 25.2 | Veh |
| D1519 | 14.0 | 24.0 | 29.0 | 300 |
| D1591 | 11.1 | 20.9 | 26.6 | 300 |
| D1593 | 11.9 | 22.6 | 27.8 | 300 |
| D2110 | 10.5 | 21.3 | 26.5 | 100 |
| D2126 | 10.2 | 21.6 | 28.0 | 100 |
| D2129 | 9.6 | 19.9 | 26.5 | 100 |

OI mice were weighed at 3 weeks of age prior to treatment, at the start of treatment at 5 weeks, and after 5 weeks of treatment with vehicle (Veh), 300 mg/kg (300), or 100 mg/kg (100) of A-395.

### Example 3: Effect of EED Inhibition on Bone

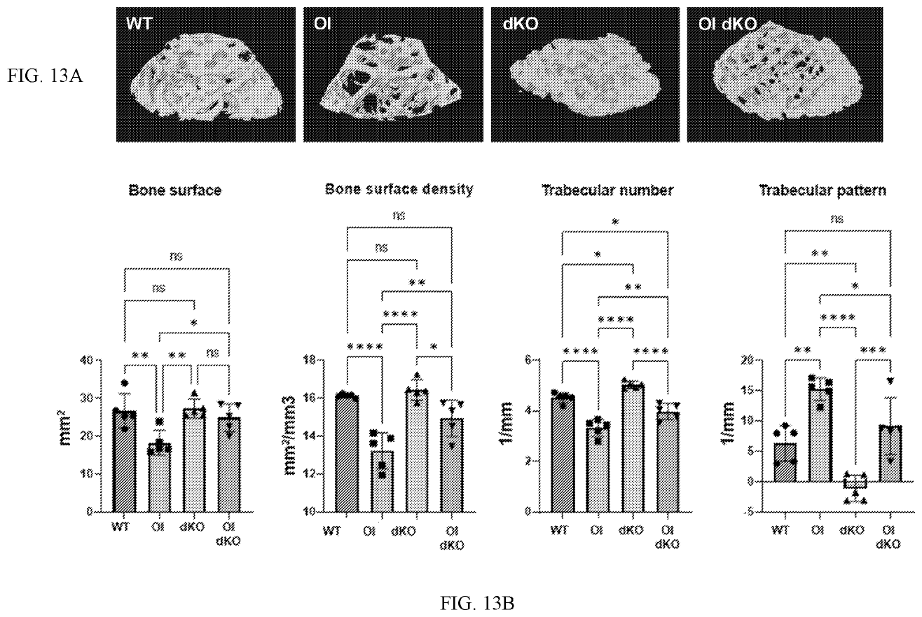

dKO mouse were crossed with the G610C mouse (Col1a2^{tm1.1Mcbr}) to generate the OI dKO mouse line. Four genotypes were assessed: (i) Ezh1wt/wt, Ezh2 wt/wt and Osx-Cre+ (wild type, WT); (ii) Ezh1KO/KO, Ezh2flox/flox, and Osx-Cre+ (dKO); (iii) Col1a2^{tm1.1Mcbr}, Osx-Cre+ (OI), and (iv) Col1a2^{tm1.1Mcbr}, Ezh1KO/KO, Ezh2flox/flox, Osx-Cre+ (OI dKO). Three male mice from each group were analyzed at 8 weeks of age. μCT analysis demonstrated robust bone formation within the femurs of dKO and OI dKO mice when compared to WT and OI mice (FIG. 13A). In addition, there was an improvement in the bone surface, bone surface density, trabecular number, and trabecular pattern in dKO and OI dKO mice compared to controls (FIG. 13B). These results demonstrated that PRC2 disruption has a beneficial effect on bone formation in OI mice.

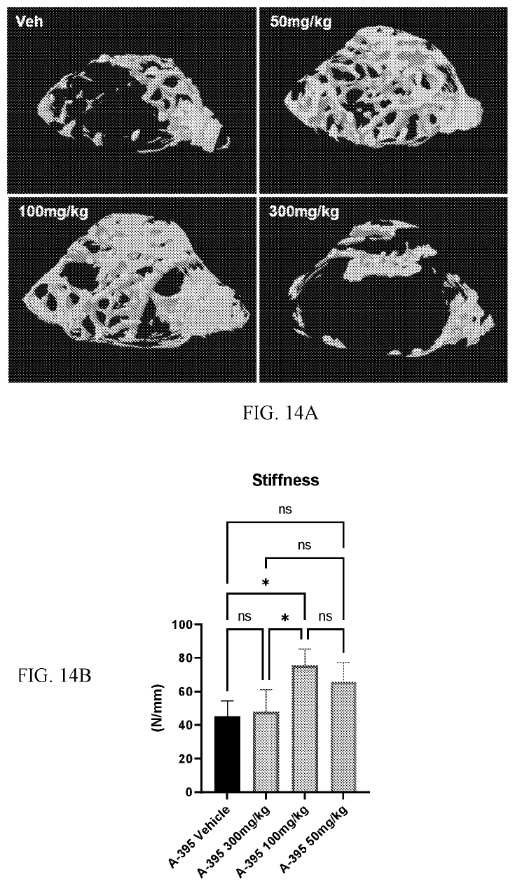

G610C mice were treat with the EED inhibitor A-395. Three 5-week-old male G610C mice were treated with either 300 mg/kg A-395 or DMSO control twice a week by subcutaneous (SQ) injection for 3 weeks. μCT analysis showed that treated mice had less bone than controls (FIG. 14A). It was then assessed if the lower bone mass was due to drug toxicity or PRC2 inhibition because at the end of the protocol the treated mice were losing weight and appeared sickly. The dose of A-395 was lowered to see if that improved the health of the mice and bone formation. Three 5-week-old male G610C mice were treated with DMSO control or either 50 mg/kg or 100 mg/kg A-395 twice a week by subcutaneous injection for 3 weeks. It was found that at both doses there was an increase in bone formation based on μCT analysis. Also, three-point bending showed improved stiffness in both the 50 and 100 mg/kg treatment groups (FIG. 14B). These results suggested that A-395 improved bone formation in OI mice.

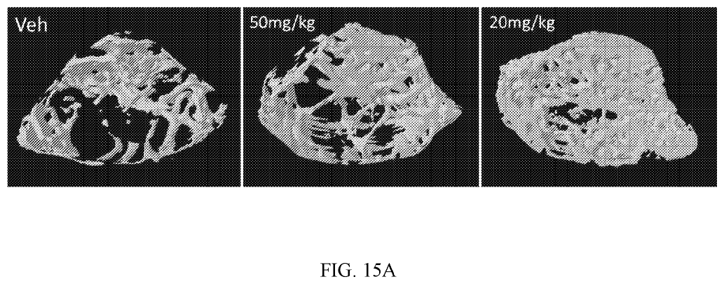

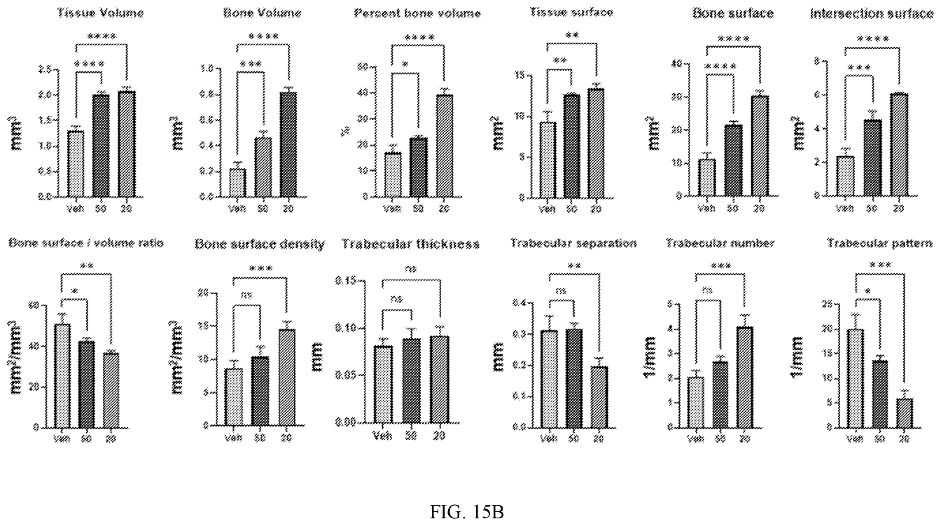

To determine if MAK683 improved bone formation in OI, three 5-week-old male G610C mice were injected SQ with 50 mg/kg MAK683 twice a week for 3 weeks. 50 mg/kg MAK683 was determined to be a similar dose to 100 mg/kg of A-395 (data not shown). μCT analysis showed that treated mice had an improvement in many bone parameters than controls (FIG. 15A). Since it was found that a lower dose of A-395 had a greater impact on bone formation, the dose of MAK683 was reduced to 20 mg/kg. Again, three 5-week-old male G610C mice were injected SQ twice a week for 3 weeks with vehicle or drug and assessed. μCT analysis showed that treated mice had improvement in essentially all bone parameters compared to controls (FIG. 15B). These results suggested that MAK683 improved bone formation in OI mice.

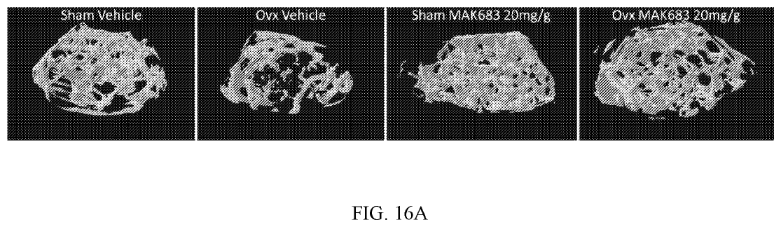

To assess whether EED inhibition could rescue low bone mass due to osteoporosis, ovariectomy (OVX) or sham (Sham) surgery on was performed 15-week-old C57BL/6 female mice with 6 animals per group. On the day of surgery, all mice were imaged using a PIXImus densitometer to measure bone density prior to OVX. Mice were allowed 7 days to recover. Mice in each group were randomized to EED inhibitor treatment or control. These groups were sham/vehicle (Sh−), sham/EED (SH+), OVX/vehicle (OV−), and OVX/EED (OV+). Again, each mouse was imaged to determine bone density after OVX prior to EED treatment. Mice were then injected with vehicle control or 20 mg/kg MAK683 SQ twice weekly for 3 weeks. After 3 weeks of treatment mice were imaged for bone density and sacrificed for analysis. μCT analysis showed that MAK683 treatment prevented bone loss in osteoporotic mice (FIG. 16).

### Example 4: Effect of EED Inhibition on OI

### Materials and Methods

One day prior to treatment 5-week-old male G610C mice were weighed to determine dosing. MAK683 was dissolved in DMSO for each treatment arm of 5, 10, 20, or 50 mg/kg for selected mice. The volume was adjusted to 100 mL for injection using corn oil. Mice were injected subcutaneously twice weekly for 3 weeks. After 3 weeks, mice were euthanized, and bone was harvested.

### Results

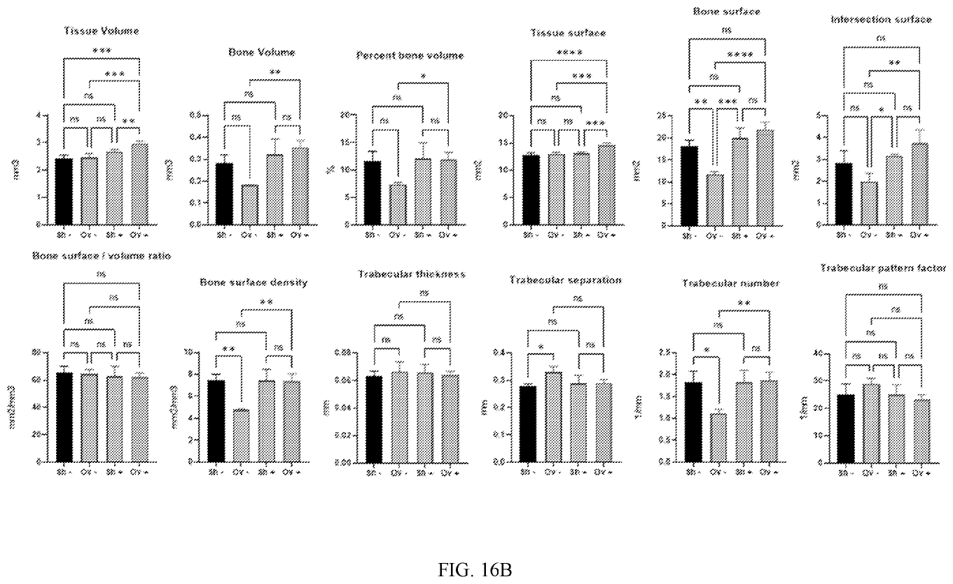

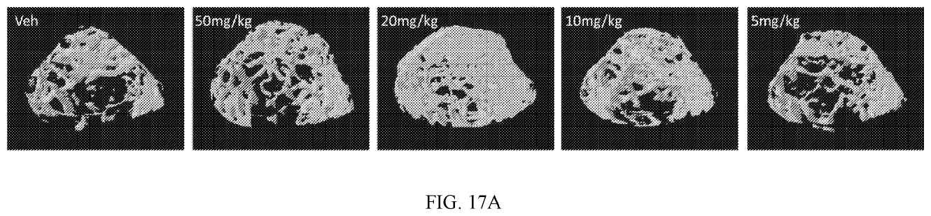

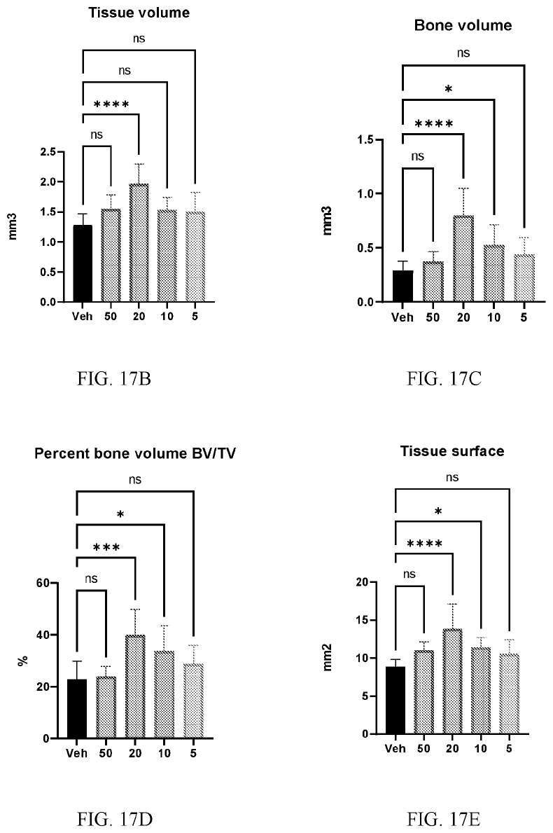

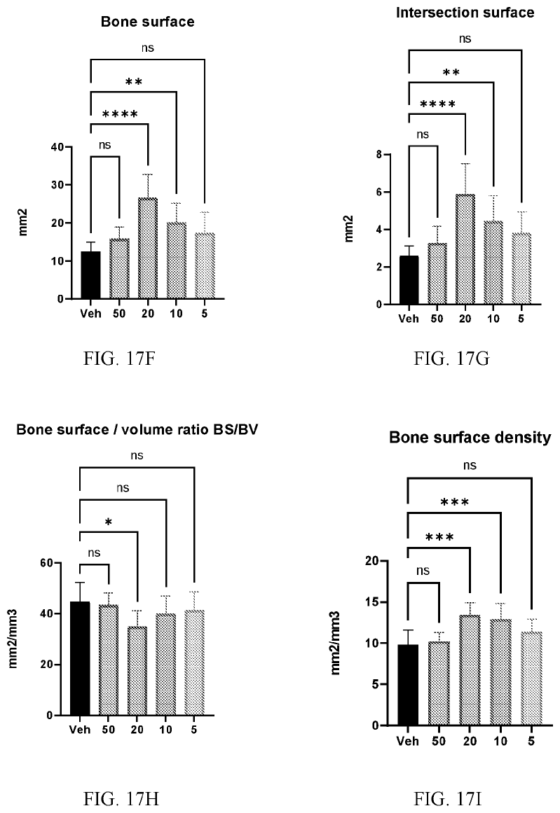

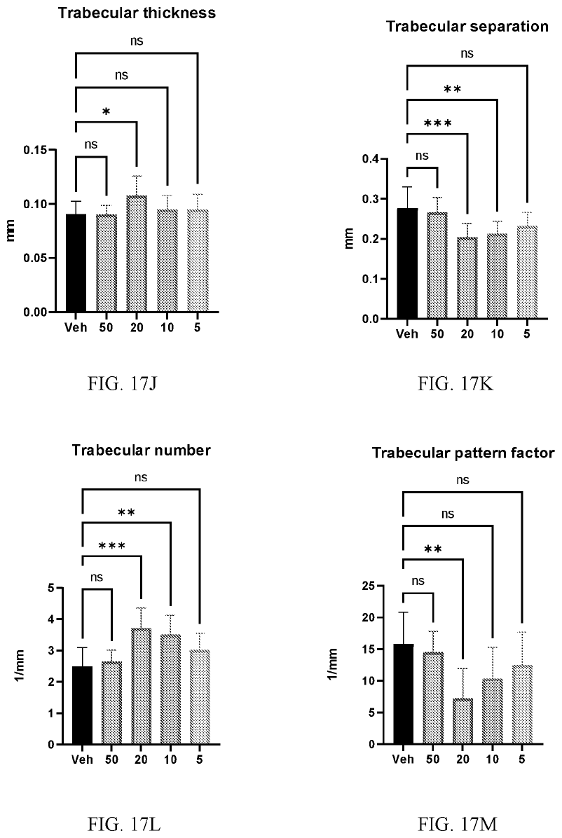

To further examine if different doses of MAK683 improved bone formation in 01, SQ nine 5-week-old male G610C mice were injected with 50, 20, 10, or 5 mg/kg MAK683 twice a week for 3 weeks. After 3 weeks, bones were harvested for analysis. μCT analysis showed that mice treated with 20 mg/kg MAK683 and 10 mg/kg MAK683 had visual improvement in bone density with possible minor improvement in doses of 50 mg/kg MAK683 and 5 mg/kg MAK683 (FIG. 17A). Mice treated with MAK683 showed improvement in many bone parameters as compared to controls (FIG. 17B-M). These results suggested that MAK683 improved bone quality in OI mice.

### Example 5: Treating Osteoporosis

A human identified as having osteoporosis is administered one or more inhibitors of an EED polypeptide selected from A-395, MAK683, EED226, EEDi-1056, EEDi-5285, EEDi-5273, FTX-6058, HJM-353, RTX-2219, and pharmaceutically acceptable salts thereof.

The administered inhibitor(s) can increase bone growth in the human by at least 5 percent as measured by BMD (e.g., as compared to a comparable human that was not administered the inhibitor).

### Example 6: Treating OI

A human identified as having OI is administered one or more inhibitors of an EED polypeptide selected from A-395, MAK683, EED226, EEDi-1056, EEDi-5285, EEDi-5273, FTX-6058, HJM-353, MRTX-2219, and pharmaceutically acceptable salts thereof.

The administered inhibitor(s) can increase bone growth in the human by at least 5 percent as measured by BMD (e.g., as compared to a comparable human that was not administered the inhibitor).

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method for promoting bone growth in a mammal having a bone disorder, wherein said method comprises administering an inhibitor of an embryonic ectoderm development (EED) polypeptide activity to said mammal, wherein bone growth increases within said mammal following said administering.

2. The method of claim 1, wherein said mammal is an adult human.

3. The method of claim 1, wherein said mammal is a juvenile human.

4. The method of claim 1, wherein said bone disorder is osteoporosis.

5. The method of claim 1, wherein said bone disorder is a genetic bone disorder.

6. The method of claim 5, wherein said bone disorder is osteogenesis imperfect (OI).

7. The method of claim 1, wherein said administering is an intravenous administration, a subcutaneous administration, or an oral administration.

8. The method of claim 1, wherein said inhibitor is selected from the group consisting of A-395 (1-(7-fluoro-2,3-dihydro-1H-inden-1-yl)-N,N-dimethyl-4-[4-(4-methylsulfonylpiperazin-1-yl)phenyl]pyrrolidin-3-amine); MAK683 (N-[(5-fluoro-2,3-dihydro-1-benzofuran-4-yl)methyl]-8-(2-methylpyridin-3-yl)-[1,2,4]triazolo[4,3-c]pyrimidin-5-amine); EED226 (n-(2-furanylmethyl)-8-[4-(methylsulfonyl)phenyl]-1,2,4-triazolo[4,3-c]pyrimidin-5-amine); EEDi-1056 (8-(2,6-dimethylpyridin-3-yl)-N-[(5-fluoro-2,3-dihydro-1-benzofuran-4-yl)methyl]-1-methylsulfonyl-2H-imidazo[1,5-c]pyrimidin-4-ium-5-amine); EEDi-5285 ((3r,4s)-1-[(1s)-7-fluoro-2,3-dihydro-1 h-inden-1-yl]-n,n-dimethyl-4-[4-[4-(methylsulfonyl)-1-piperazinyl]phenyl]-3-pyrrolidinamine); EEDi-5273 (15-[(5-fluoro-2,3-dihydro-1-benzofuran-4-yl)methylamino]-9-propan-2-yl-5-(trifluoromethyl)-4,9,12,14,16-pentazatetracyclo[9.6.1.02,7.014,18]octadeca-1(17),2,4,6,11(18),12,15-heptaen-10-one); and pharmaceutically acceptable salts thereof.

9. The method of claim 1, wherein said bone growth increased within about 3 weeks of said administering.

10. The method of claim 1, wherein said bone growth increased within about 3 days of said administering.

11. The method of claim 1, wherein said bone growth increased by at least 5 percent as measured by bone mineral density (BMD) as compared to a comparable mammal not administered said inhibitor.

12. The method of claim 1, wherein said method comprises identifying said mammal as having said bone disorder.

13. The method of claim 1, wherein said method comprises identifying said mammal as being in need of increased bone growth.

14. The method of claim 1, wherein said method comprises identifying said mammal as being in need of said inhibitor for promoting bone growth.

15. The method of claim 1, wherein said method comprising administering said inhibitor to said mammal from about once per day to about once per month.

16. The method of claim 1, wherein said method comprising administering said inhibitor to said mammal from about once per day to about once per week.

17. The method of claim 1, wherein said method comprising administering from about 5 mg of said inhibitor per kg of body weight of said mammal to about 300 mg of said inhibitor per kg of body weight of said mammal.

18. The method of claim 1, wherein said method comprising administering from about 50 mg of said inhibitor per kg of body weight of said mammal to about 100 mg of said inhibitor per kg of body weight of said mammal.

19. The method of claim 1, wherein said mammal does not have cancer.

20. (canceled)

21. The method of claim 1, wherein said inhibitor is selected from the groups consisting of FTX-6058 ((S)-12-fluoro-4-(2-methylpyridin-3-yl)-7a,8,13,14-tetrahydro-7H-[1,2,4]triazolo[4′,3′:1,6]pyrido[3,2-b]benzofuro[4,3-fg][1,4]oxazonine); HJM-353; MRTX-2219 ((4S)-8-{4-[(dimethylamino)methyl]-2-methylphenyl}-5-{[(5-fluoro-2,3-dihydro-1-benzofuran-4-yl)methyl]amino}imidazo[1,2-c]pyrimidine-2-carbonitrile)); and pharmaceutically acceptable salts thereof.
